(19) United States
(12) Patent Application Publication
(10) Pub. No.: US 2008/0039532 A1
(43) Pub. Date: Feb. 14, 2008

(54) Compounds For Specific Viral Target

(76) Inventors: Dominique Bridon, San Francisco, CA (US); Nathalie Bousquet-Gagnon, Lachine (CA); Xicai Huang, Belle Mead, NJ (US); Omar Quraishi, Lachine (CA)

Correspondence Address: FISH & RICHARDSON PC, P.O. BOX 1022, MINNEAPOLIS, MN, 55440-1022, US

(21) Appl. No.: 11/579,929

(22) PCT Filed: May 6, 2005
(86) PCT No.: PCT/CA05/00689

§ 371 Date: Sep. 10, 2007

Related U.S. Application Data
(60) Provisional application No. 60/569,219, filed on May 6, 2004.

(51) Int. Cl. A61K 47/48; A61K 47/42; C07K 14/00

(52) U.S. Cl. 514/773; 530/324

(57) ABSTRACT

The present application relates to a compound having a formula 1: B—R1-R2-M wherein B is a binding element for recognizing and binding a target; R1 is a first group of atoms for reacting with a functionality of the target so as to form a covalent bond with the target; R2 is a second group of atoms; R1 and R2 being such that the formation of the covalent bond between R1 and the target generates cleavage of the bond between R1 and R2 so as to free R2-M; and M is selected from the group consisting of a hydrogen atom and a pharmaceutically acceptable moiety. Alternatively, R1 and R2 can be inverted to form the formula II: B—R2-R1-M and being such that the formation of the covalent bond between R1 and the target generates cleavage of the bond between R1 and R2 so as to free R2-B.

### FIELD OF THE INVENTION

The present invention relates to compounds for specific viral target and related compositions and methods thereof, and more specifically to virus entry inhibitors and anti-fusiogenic compounds.

### BACKGROUND OF THE INVENTION

In many therapeutic fields, the strategy of treatment is based on inhibitors or ligands that reversibly bind to their specific viral targets. These inhibitors and ligands alternate between the forms where they are bound to specific targets and their free forms. When free, they may be subject to enzymatic degradation and/or rapid kidney excretion that results in loss of therapeutic efficacy. There is a need for covalently and irreversibly attached inhibitors or ligands to their specific viral targets in order to extend their therapeutic effect.

### SUMMARY OF THE INVENTION

In accordance with the present invention, there is now provided a compound having the formula I:
B—R1-R2-M I
wherein:
B is a binding element for recognizing and binding a target;
R1 is a first group of atoms for reacting with a functionality of the target so as to form a covalent bond with the target;
R2 is a second group of atoms; R1 and R2 being such that formation of the covalent bond between R1 and the target generates cleavage of the bond between R1 and R2 so as to free R2-M; and
M is selected from the group consisting of a hydrogen atom and a pharmaceutically acceptable moiety.

In accordance with the present application, there is also provided a compound having the formula II:
B—R2-R1-M II
wherein:
B is a binding element for recognizing and binding a target;
R1 is a first group of atoms for reacting with a functionality of said target so as to form a covalent bond with said target;
R2 is a second group of atoms; R1 and R2 being such that formation of the covalent bond between R1 and said target generates cleavage of the bond between R1 and R2 so as to free B—R2; and
M is a pharmaceutically acceptable moiety.

The moiety of this compound may comprise a bulky agent, preferably selected from the group consisting of a drug or therapeutic agent, a protein, a molecule, a particle, a polymer, a liposome and a cell, more preferably a serum protein (endogenous, recombinant or genomic), and even more preferably the bulky agent is serum albumin.

In a preferred embodiment of the present invention, R1 is of the formula III:
X—C(Y) III
wherein

X is absent or selected from the group consisting of alkyl groups, and substituted or unsubstituted phenyl groups; and

Y is selected from the group consisting of sulfur, oxygen, phosphorus and nitrogen, preferably from the group consisting of sulfur and oxygen.

As an example, when Y is oxygen, C(Y) is carbonyl.

In the present invention, a substituted phenyl group is preferably a phenyl group bearing at least one substituent such as halogen, NO₂, SO₂NH₂, SO₂NHF, CF₃, CCl₃, CBr₃, C═N, SO₃H, CO₂H, CHO, NHR, OH, NHCOCH₃, OCH₃, CH₃ and CH₂CH₃.

In a preferred embodiment of the present invention, R2 is selected from the group consisting of oxygen, acetal, hemiacetal, phosphoacetal, sulfur, alkoxy, thioalkoxy, hydroxyamino derivatives, either substituted or unsubstituted phenoxy, thiophenoxy, and aminophenoxy derivatives. In the present invention, a substituted phenoxy, thiophenoxy or aminophenoxy contains a phenyl group bearing at least one substituent such as halogen, NO₂, SO₂NH₂, SO₂NHF, CF₃, CCl₃, CBr₃, C═N, SO₃H, CO₂H, CHO, NHR, OH, NHCOCH₃, OCH₃, CH₃ and CH₂CH₃.

In another embodiment of the present invention, R1R2 is such as to include a reactive functional group selected from the group consisting of alkyl ester, aryl ester, alkyl thioester, aryl thioester, phosphoester, ortho ester, imidate, mixed anhydride, disulphide, amide and thioamine. The reactive group can also include an aromatic moiety such as, but not limited to, a substituted or unsubstituted phenyl group as described above. Preferably, the reactive functional group formed by R1R2 is stable in an aqueous enviromnent.

In one embodiment of the present invention, the binding element is selected from the group consisting of an organic compound, an amino acid sequence, a peptide, a protein, a nucleic acid sequence, a small molecule, a mimetic thereof and a combination thereof.

In one embodiment of the present invention, the viral target is selected from the group consisting of a virus, a viral antigen, a receptor on an infected cell, a viral peptide, an infected cell, a viral protein expressed at the surface of an infected cell, fragments thereof or specific regions thereof.

In a preferred embodiment of the present invention, the virus is selected from the group consisting of Human Immunodeficiency Virus (HIV-1 and 2), Respiratory Syncytial virus (RSV), influenza virus, human Papilloma Virus (HPV), Ebola, dengue, rubella, Epstein Barr, Hepatitis, HTLV-1 and 2, Semliki Forest Virus (SFV), Measle Virus (MeV), yellow fever, Japanese encephalitis, West Nile and tick-borne encephalitis (TBE) viruses. In this embodiment, the binding element can be, but is not limited to, a binding element having a binding affinity for a region of gp41 glycoprotein or analog and equivalent thereof of the virus.

In a more preferred embodiment of the present invention, the binding element has an amino acid sequence selected from the group SEQ ID NOS:1 to 19 and the virus is HIV.

In another embodiment of the present invention, the moiety comprises a therapeutic agent. This therapeutic agent may be selected from the group consisting of drugs, protease inhibitors, antiproliferative agents, antisense oligonucleotides, antiviral agents, virus entry inhibitors and anti-fusiogenic agents.

In accordance with the present invention, there is also provided a compound of the present invention further comprising a linker L1 between B and R1 when the compound is of the configuration B—R1-R2-M. Additionally or alternatively, a linker L2 can be present between R2 and M.

In accordance with the present invention, there is also provided a compound of the present invention further comprising a linker L2 between B and R2 when the compound is of the configuration B—R2-R1-M. Additionally or alternatively, a linker L1 can be present between R1 and M. Typically, the linkers are of about 1-20 atoms in length, which atoms may be carbon, nitrogen, oxygen, sulfur, phosphorus and the like. The linkers may be alkylene groups, generally of about 2-16 carbon atoms, more generally of about 1-25 carbon atoms; polyoxyalkylene groups, where the alkylene groups will be of 2-3 atoms, and having about 1-8 units and preferably about 1-6 units; an amino acid including alpha and omega amino acids, or oligonucleotide having about 1-8 amino acids and preferably about 1-6 amino acids, where the amino acids may be polar or non-polar, charged or uncharged, aliphatic, alicyclic, aromatic or heterocyclic, naturally occurring or synthetic, dextrogyre (D) or levogyre (L). In the present invention, it is preferred that the linker has the formula —NH—(CH₂)ₙ—C(O)—, where n is an integer varying from 1 to 25, more preferably, the linker is chosen from —NH—(CH₂)₅—C(O)— and —NH—CH₂—C—(O)—.

In accordance with the present application, there is provided a method for modulating an activity of a viral target in a subject comprising administering to said subject the compound of the present invention, alone or in association with a pharmaceutically acceptable carrier; the binding element having a binding affinity for a region of the viral target involved in the activity of the viral target, whereby the bonding of the compound to the region of the viral target results in the interruption of the activity of the target.

In accordance with the present application, there is also provided an antiviral composition for modulating an activity of a viral target in a subject comprising a compound of the present invention in association with a pharmaceutically acceptable carrier; said binding element having a binding affinity for a region of the viral target involved in the activity of a membrane fusion process of cell infection of a virus, whereby the bonding of said compound to said region of the target results in the interruption or reduction of the activity of the target.

The modulation of the activity in the present application is preferably, but not limited to, an interruption or a reduction of the activity.

In one embodiment of the present application, the activity is a membrane fusion process of cell infection of a virus.

In accordance with the present application, there is further provided the use of a compound of the present invention for the manufacture of a medicament for use in an antiviral treatment of a subject. More particularly, the subject in need of the antiviral treatment is infected by a virus selected from the group consisting of Human Immunodeficiency Virus (HIV-1 and 2), Respiratory Syncytial virus (RSV), influenza virus, human Papilloma Virus (HPV), Ebola, dengue, rubella, Epstein Barr, Hepatitis, HTLV-1 and 2, Semliki Forest Virus (SFV), Measle Virus (MeV), yellow fever, Japanese encephalitis, West Nile and tick-borne encephalitis (TBE) viruses.

### BRIEF DESCRIPTION OF THE DRAWINGS

FIG. 1 illustrates HPLC of C34:N36 (1655/1722) dimer;

FIG. 2 illustrates HPLC of C34:N36 (1655/1723) dimer;

FIG. 3 illustrates HPLC of C34:N36 (1646/1722) dimer;

FIG. 4 illustrates LC/MS identification of covalent N36:C34 dimer; and

FIG. 5 illustrates native PAGE of N36/C34 complexes.

### DETAILED DESCRIPTION OF THE INVENTION

In order to ensure a complete understanding of the invention, definitions of the terms used in the description are first provided.

Binding Element: The binding element comprises a region that has a specific binding affinity for a complementary region on a viral target and may be embodied as an organic compound, an amino acid sequence, a peptide, a protein, a hormone, an antibody, an antigen, a nucleic acid sequence, a mimetic or any combination of the above.

Viral Target: A viral target is an entity for which it is desirable to modulate the activity or that interacts with another entity to provide an activity that is desirable to modulate. The viral target may be, but is not limited to, a virus, a viral antigen expressed on the surface of an infected cell, a ligand specific to a virus or a viral antigen of a surface receptor on an infected cell, an infected cell surface receptor, a peptide, an infected cell or membrane thereof, a viral protein expressed at the surface of an infected cell, such as gp41, fragments thereof or specific regions thereof (ie: N-heptad repeat or C-heptad repeat of gp-41).

Functionality: Functionality is a group of atoms that represents a potential reaction site on the viral target. Functionality includes but not limited to carboxy, amino, thiol and hydroxyl group. Amino group is preferred and may be provided by a lysine, arginine, asparagine or glutamine residue or the free N-terminus of a peptide or protein.

Linker: A linker is optionally used between the binding element and the reactive group and/or between the reactive group and the moiety. The length of the linker may vary in order to allow the binding element to bind the region of the viral target for which it has an affinity and concurrently allow the reactive group to react with a functionality of the viral target and form a covalent bond. Activity assays and competition binding assays are useful to determine the appropriate length of the linker.

Pharmaceutically acceptable Moiety: A pharmaceutically acceptable moiety is intended to be a moiety that would be of acceptable use in a subject for administration in vivo. Such moiety can be a hydrogen atom, or may comprise a therapeutic agent, a pro-drug and/or a bulky agent. The bulky agent may be naturally occurring (endogenous), or be of synthetic, genomic or recombinant source, such as serum proteins, more particularly, serum albumin. The moiety may also comprise an attaching group covalently attached to the bulky agent. Also, the attachment group may comprise a linking group in order to distance the bulky agent from the reactive group and the binding element if desired to avoid steric hinderance. Examples of therapeutic agents suitable for the present invention are selected from, but not limited to, peptides, small molecules, drugs, antisense oligonucleotides, antiviral agents, virus entry inhibitors and anti-fusiogenic agents.

To Bind: It is intended for the purpose of the present invention that binding involves electronic, hydrophobic, electrostatic or van der Waals attraction between two molecules, two amino acid sequences, two nucleic acid sequences or else. The strength of such attraction is usually called the binding affinity. Binding is a reversible interaction being in a dynamic equilibrium between a bound state and an unbound state. According to the present invention, such binding occurs between the binding element and the viral viral target.

To Bond: It is intended for the purpose of the present invention that bonding involve a chemical reaction and a rearrangement in order to form a covalent bond between two molecules. Bonding is an irreversible interaction. According to the present invention, such bonding occurs between the reactive group (R1) and the viral target.

Activity of the viral Target: An activity of the viral target includes one of the intrinsic activities of the viral target. For example, it may be any process involved during cell infection of a virus or a virus-infected cell (i.e. membrane fusion process).

Pharmaceutically Acceptable Carrier: A pharmaceutically acceptable carrier may in the form of pill, gel capsule, aqueous solution or the like and has a purity and/or an osmolarity acceptable by the subject.

Subject: A subject is a mammal or a human.

gp41 glycoprotein and gp41 glycoprotein analogs: gp41 glycoprotein is a protein involved in the virus entry step or the membrane fusion step of the cell infection process of a virus such as HIV, SIV, RSV, HPV (HPIV) and MeV. gp41 glycoprotein as disclosed and claimed includes truncations, deletions and/or insertions thereof. Deletions consist of the removal of one or more amino acid residues from the gp41 glycoprotein, and may involve the removal of a single contiguous portion of the amino acid sequence or multiple portions. Insertions may comprise single amino acid residues or stretches of residues and may be made at the carboxy or amino terminal end of the sequence or at a position internal to the sequence.

gp41 glycoprotein analogs are proteins comprises in peptide regions of virus other than HIV that correspond to the gp41 glycoprotein region, as well as truncations, deletions and/or insertions thereof.

A more specific region of gp41 targeted in accordance with the present invention is the N-heptad repeat or the C-heptad repeat.

Fragment: Fragment is a portion of the full-length sequence of peptide, DNA or molecule, which has retained some of the properties of the complete peptide, DNA or molecule in order to play its role for achieving the present invention. Thus, it is intended that the fragment disclosed is able to bind the viral target.

Pro-drug: A pro-drug is a compound that undergoes chemical and/or structural modifications in vivo, enzymatically or chemically, that confers an activity to the molecule distinct from that of the original compound, such as an anti-viral activity.

### DETAILED DESCRIPTION OF THE INVENTION AND ITS PREFERRED EMBODIMENTS

The present invention relates to a compound having the formula I: B—R1-R2-M or the formula II: B—R2-R1-M.

B is a binding element that has a binding affinity for a viral target as previously defined. According to the present invention, the binding element is able to recognize and bind a desired viral target. Such binding element and viral target can be the ones already known in the art. Alternatively, a binding element for a known specific viral target can be determined by the method of screening disclosed in the International application WO 99/24075 that is incorporated herein by reference.

R1 is a first reactive group being able to react with a functionality of the viral target so as to form a covalent bond with the viral target as previously defined. According to a preferred embodiment of the invention, R1 is of the formula III:
X—C(Y) III
wherein

X is absent or selected from the group consisting of aliphatic alkyl groups and cyclic alkyl groups.

Y is selected from the group consisting of sulfur, oxygen, phosphorus and nitrogen, preferably from the group consisting of sulfur and oxygen.

R2 is a group of atoms being such that the formation of the covalent bond between R1 and the viral target generates cleavage between R1 and R2 as previously defined. R2 is preferably selected from the group consisting of oxygen, acetal, hemiacetal, phosphoacetal, sulfur, alkoxy, thioalkoxy, hydroxyamino derivatives, either substituted or unsubstituted phenoxy as previously defined, thiophenoxy, aminophenoxy derivatives, and substituted or unsubstituted phenyl group. In the present invention, a substituted phenyl group is preferably a phenyl group bearing substituents such as halogen, NO₂, SO₂NH₂, SO₂NHF, CF₃, CCl₃, CBr₃, C═N, SO₃H, CO₂H, CHO, NHR, OH, NHCOCH₃, OCH₃, CH₃ and CH₂CH₃.

In another embodiment of the present invention, R1R2 is such as to include a reactive functional group selected from the group consisting of alkyl ester, aryl ester, alkyl thioester, aryl thioester, phosphoester, ortho ester, imidate, mixed anhydride, disulphide, amide and thioamine. The reactive group can also include an aromatic moiety such as, but not limited to, a substituted or unsubstituted phenyl group as described above. Preferably, the reactive functional group formed by R1R2 is stable in an aqueous environment.

However, it is understood herein that one skilled in the art would easily recognize what other reactive groups, R1, R2 and combinations of R1R2 could be suitable for the purpose of the present invention.

M may be hydrogen or a pharmaceutically acceptable moiety as previously defined.

Additionally, the compound of the present invention may comprise a linker L1 between B and R1 and/or a linker L2 between R2 and M. The purpose of the linkers is to space out the elements of the compound in order to allow the reactive group to reach a compatible functionality of the viral target so as to form a covalent bond with it when the binding element is already bound to the viral target. Moreover, in the compound of the formula B—R2-R1-M, when the moiety comprises a bulky agent, the linker between M and R1 prevents steric hindrance with the covalent bonding of the reactive group to the viral target and/or the binding interaction between the binding element and the viral target.

By means of “compatible functionality”, it is intended a functionality that can react with the reactive group and form a covalent bond with it. A large array of linkers is commercially available or may be synthesized, and the linker of the present invention is not limited to a specific one. To determine the more appropriate linker to use, different linkers with varying lengths and flexibilities are assayed.

The binding element “B” includes without limitation the following: 5-helix, NCCG-gp41, N36 Mut(e.g), DP-107, T-21, N36, enfuvirtide, T-20, Fuseon, DP-178, pentafuside, T-1249, ADS-J1, SC34EK, IQN17, D10-PX-2K, IQN23, C14linkmid, C34coil, (Cys)C34-GCN4(Cys)GCN4, T-649, C14, SJ-2176, scC34, sC34, C34, p38, p26, siamycin I and II, ADS-J2, ADS-J1, N-36-E, S-29-I, SPC-3, CLIV, AMD-070, KRH-1636, KRH-1120, CXCR4 blocker or antagonist, T-134, T-140, AMD-8664, HIV-1 Tat analogs, ALX40-4C, AMD-3100, T-22, 5,12-Tyr 7-Lys polyphemusin II, TJN-151, baicalin, AM-1401, NSC-651, Conocurvone, DAPTA, D-Ala1 peptide T-amine, SCH—C, SCH-D, TAK-220, SCH-350, CCR5 antagonist, peptide T, UCB-35, EGCG, Epigallocathechin Gallate, Carraguard, lambda-carrageenan, curdlan sulfate, OKU-40, OKU-41, Zintevir, cosalane analog, dextrin-2-sulfate, scyllatoxin analog, CDR2-like loop, HIV p7 inhibitors, michellamine A, B and F.

### DESCRIPTION OF A FIRST PREFERRED EMBODIMENT OF THE INVENTION

According to a first preferred embodiment, the reactive group is R1-R2 and is oriented in the formula of the compound as follow B—R1-R2-M (I). In this case, the M is released with R2 and the binding element B remains covalently attached to the viral target through the bonding of R1. The nature of the binding element is such that it modulates at least one specific activity of the viral target. Examples of this embodiment are provided below.

An application of the first preferred embodiment is found for stopping, reducing or preventing viral infection. In such a case, the viral target is a virus and the binding element has a binding affinity region to that virus, for example to gp41. Examples of binding elements for gp41-related viruses and analogs thereof are illustrated below in Table 1. By binding such a region and staying covalently attached to such a region by means of the reactive group of the compound, the virus entry and the membrane fusion processes are interrupted and the HIV infection is inhibited on a permanent basis. This is unlike for non-covalent binding elements which dissociate from their binding region according to their intrinsic dissociation constant (Kd). They are subject to enzymatic degradation and plasma clearance through the kidneys. Their anti-viral activities decline over time due to the emergence of viral resistance due, at least in part, to the virus ability to mutate as well as fluctuating in vivo concentrations of the compounds. These binding elements have been found to be selective for binding to a region of gp41 glycoprotein that is involved in the entry and more specifically in the membrane fusion process responsible for the penetration of HIV into an uninfected cell. This mechanism of action of the compound can be applied for inhibiting the cell infection process of other viruses such as, but not limited to, Respiratory Syncytial virus (RSV), influenza virus, human Papilloma Virus (HPV), Ebola, dengue, rubella, Epstein Barr, Hepatitis, HTLV-1 and 2, Semliki Forest Virus (SFV) and Measle Virus (MeV).

A number of viruses from different families are now known to have fusion proteins with strikingly similar structural features, such as an orientation perpendicular to the membrane (as in ‘spikes’), the presence of amino-terminal or amino-proximal fusion peptides, and the formation of a characteristic post-fusion hairpin structure built upon a three-stranded coiled coil of alpha-helices. Such proteins are present in orthomyxoviruses, paramyxoviruses, retroviruses and filoviruses, and are designated class I viral fusion proteins. For example, the core of the fusion-active state of gp41 shows similarity to the proposed fusiogenic structures of envelope fusion proteins from influenza (Bullough, P. A. et al (1994) Nature, 371, 37-43), Moloney murine leukemia virus (Fass, D., and Kim, P. S. (1995) Curr. Biol., 5, 1377-1383; Fass, D. et al. (1996) Nat. Struct. Biol., 3, 465-469), simian parainfluenza virus 5 (Baker, K. A. et al. (1999) Mol. Cell., 3, 309-319), Ebola virus (Malashkevich, V. N. et al. (1999) Biochemistry, 96, 2662-2667), and simian immunodeficiency virus (Caffrey, M. et al. (1998) EMBO J., 17, 4572-4584; Yang, Z. et al. (1999) J. Struct. Biol. 126, 131-144; Maleshkevich, V. N. et al. (1998) Biochemistry, 95, 9134-9139).

The fusion machinery of flaviviruses and alphaviruses have completely different structural features, and thus the corresponding proteins are therefore designated class II viral fusion proteins, yet the proposed fusion mechanisms are similar to those for the class I fusion proteins (Bressanelli, S. et al. (2004) EMBO J. 23, 728-738; Modis Y. et al. (2004) Nature 427, 313-319). Flaviviruses are small, icosahedral enveloped viruses that constitute a genus within the family Flaviviridae which also includes the genera pestivirus and hepacivirus (human hepatitis C viruses). Several flaviviruses are important mosquito- or tick-borne human pathogens, such as the yellow fever, dengue, Japanese encephalitis, West Nile and tick-borne encephalitis (TBE) viruses. Mature virions contain three proteins, designated C (capsid), E (envelope) and M (membrane) proteins. The E protein is the major constituent of the virion surface and has the dual function of binding cell receptors and mediating low pH-triggered membrane fusion in the endosome. Virus assembly takes place in the endoplasmic reticulum and first leads to the generation of fusion-incompetent, immature virions in which the E protein forms stable heterodimeric complex with precursor of M (prM). Immature virions are transported through the cellular endocytotic pathway and, shortly before their release, prM is cleaved by furin or a related protease in the trans-Golgi network to generate fusion-competent mature infectious virions.

TABLE 1

| | |
|---|---|
| | Amino acid sequence |
| SEQ ID NO: | (one letter code) |

| | |
|---|---|
| 1 | YTSLIHSLIEESQNQQEKNEQELLELDKWASLWNWF |
| 2 | YTDLIHSLIEESQNQQEKNEQELLELDKWASLWNWF |
| 3 | NNLLRAIEAQQHLLQLTVWGIKQLQARILAVERYLKDQ |
| 4 | YTGLIYRLIEESQTQQEKNELLELDKWASLWNWF |
| 5 | WMEWDREINNYTSLIHSLIEESQNQQEKNEQELL |
| 6 | WXEWDRKIEEYTKKIKKLIEESQEQQEKNEKELK |
| 7 | WXEWDRKIEEYTKKIEELIKKSQEQQEKNEKELK |
| 8 | WEEWDKKIEEYTKKIEELIKKSEEQQKKNEEELKK |
| 9 | WMEWDREINNYTSLIHSLIEESQNQQEKNEQELLEL |
| 10 | WMEWDREINNYTSLIHSLIEESQNQQEKNEQELLK |
| 11 | WMEWDREIKNYTSLIHSLIEESQNQQEKNEQELL |
| 12 | WMEWDREINNYTSLIHSLIEESQNQQERNEQELL |
| 13 | WMEWKREINNYTSLIHSLIEESQNQQERNEQELL |
| 14 | WMEWDREKNNYTSLIHSLIEESQNQQERNEQELL |
| 15 | WQQWDEKVRNYSGVIFGLIEQAQEQQNTNEKSLL |
| 16 | WQEWDQQINNVSSIIYEEIQKAQVQQEENEKKLL |
| 17 | WQQWERQVRFLDANITKLLEEAQIQQEKNMYELQ |
| 18 | WQEWEQQVRYLEANISEQLERAQIQQEKNTYELQ |
| 19 | WQEWESQITALEGNISTTLVKAYEQEQKNMDTYQ |

*where X is any amino acid

In the case of the antiviral use for the HIV-1 virus, a known inhibitor of viral fusion called T-20 (SEQ ID NO:1) can be used. The viral target is the envelop protein gp41 of the HIV-1 virus that is known to have a high binding affinity for T20 (SEQ ID NO:1) or gp41 expressed on an infected cell surface. The reactive functional groups on the viral target are the epsilon amine of the lysine residues that are located in the vicinity of the T-20/gp41 binding site(s), such as N-heptad repeat corresponding T-20 sequence in gp41 at C-heptad repeat at or near gp41 transmembrane domain. Upon binding of T-20 with the viral envelop protein, and if the molecular distance is appropriate, the reactive group reacts with functional reactive group to yield a covalent peptide bond that attaches the binding element to the target molecule. The covalent attachment of T-20 (SEQ ID NO:1) to its target binding site(s) prevents the molecular fusion event to occur on a permanent basis and thus prevents the virus from infecting the uninfected cell.

Peptide analogs of native C34 (SEQ ID NO:5) are also included in accordance with the present invention. Such analogs include one or more amino acid modifications of the native peptides at the position identified in formula IV below as X on the basis that these residues are solvent exposed and not involved in binding with N36 during the six-helix bundle formation (Akira Otaka et al., Angew. Chem. Int. Ed. 2002, 41, No. 16, 2937-2940). Accordingly, any amino acid can be substituted at one or more of these positions without affecting the binding affinity of the analog peptide with N36. Preferred analogs only contain one Lysine and such Lysine residue is substituting any amino acid residue at position number 1, 4, 5, 8, 11, 12, 15, 18, 19, 22, 25, 26, 29, 32 and 33 in formula IV below creating one reactive group capable of being chemically modified with R1-R2-M (i.e. substituted phenyl). The preferred position for such a Lysine residue is at position 1, 5 and 8, most preferably at position 5. More preferably, the Lysine residue at position 28 in the native sequence of C34 is replaced by a less reactive amino acid, such as Arginine.
W¹X²X³W⁴D⁵X⁶X⁷I⁸X⁹X¹⁰Y¹¹T¹²X¹³X¹⁴I¹⁵X¹⁶X¹⁷L¹⁸I¹⁹X²⁰X²¹S²²X²³X²⁴Q²⁵Q²⁶X²⁷X²⁸N²⁹X³⁰X³¹E³²L³³X³⁴ IV

Preferably, peptide analogs are modified at their N-terminus with one acetyl or at least one cysteic acid (1, 2 or more) to increase their solubility.

Table 2 illustrates compounds of the present invention wherein the binding element is SEQ ID NO:1.

TABLE 2

| | |
|---|---|
| | SEQ ID NO: 20 |
| 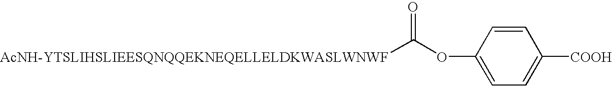 | |
| | SEQ ID NO: 21 |
| 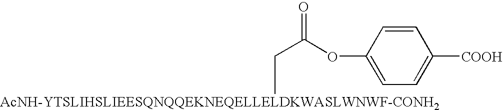 | |
| | SEQ ID NO: 22 |
| 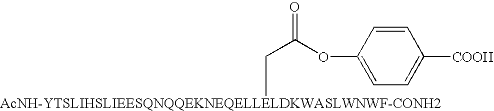 | |
| | SEQ ID NO: 23 |
| 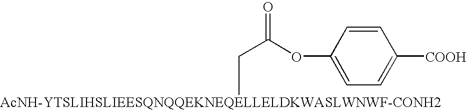 | |
| | SEQ ID NO: 24 |
| 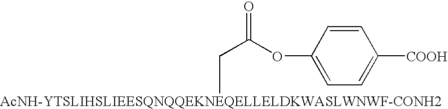 | |
| | SEQ ID NO: 25 |
| 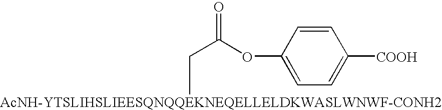 | |
| | SEQ ID NO: 26 |
| 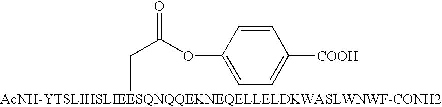 | |
| | SEQ ID NO: 27 |
| 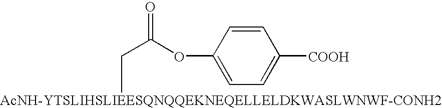 | |
| | SEQ ID NO: 28 |
| 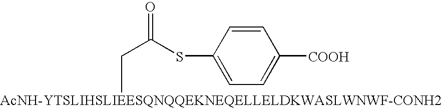 | |
| | SEQ ID NO: 29 |
| 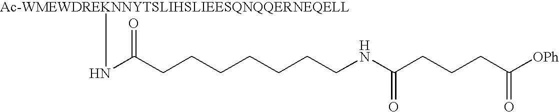 | |
| | SEQ ID NO: 30 |
| 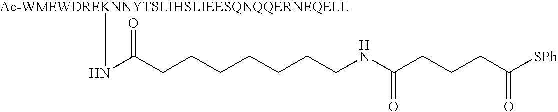 | |
| | SEQ ID NO: 31 |
| 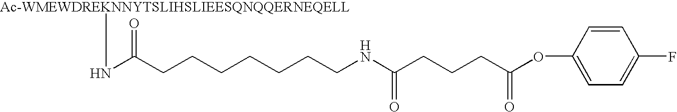 | |
| | SEQ ID NO: 32 |
| 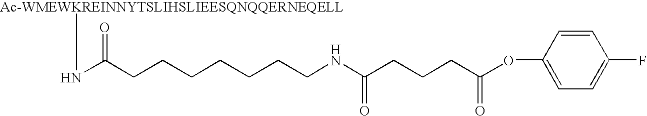 | |
| | SEQ ID NO: 33 |
| 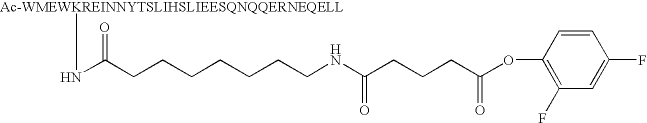 | |
| | SEQ ID NO: 34 |
| 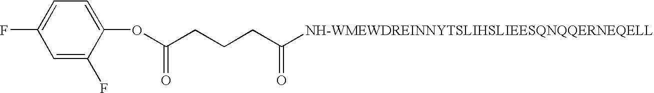 | |
| | SEQ ID NO: 35 |
| 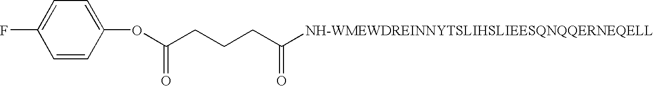 | |
| | SEQ ID NO: 36 |
| 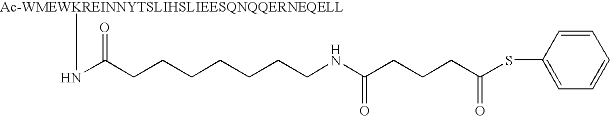 | |
| | SEQ ID NO: 37 |
| 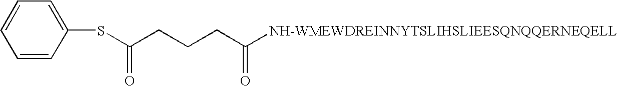 | |
| | SEQ ID NO: 38 |
| 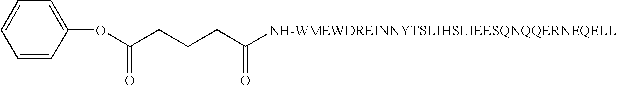 | |
| | SEQ ID NO: 39 |
| 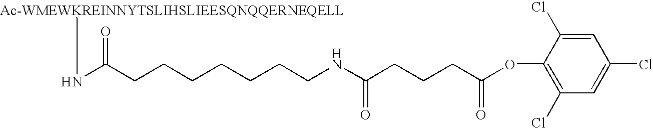 | |
| | SEQ ID NO: 40 |
| 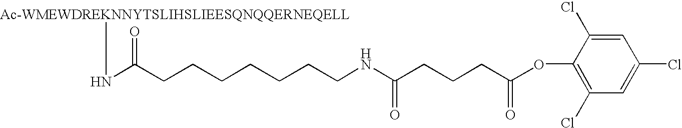 | |
| | SEQ ID NO: 41 |
| 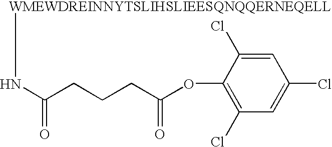 | |
| | SEQ ID NO: 42 |
| 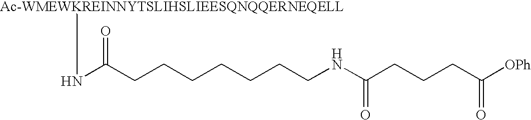 | |
| | SEQ ID NO: 43 |
| 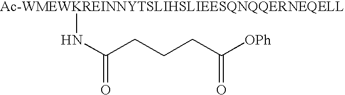 | |
| | SEQ ID NO: 44 |
| 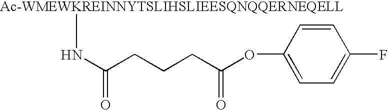 | |
| | SEQ ID NO: 45 |
| 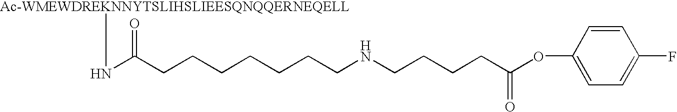 | |
| | SEQ ID NO: 46 |
| 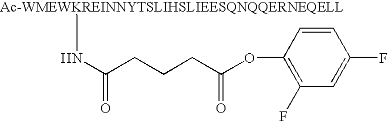 | |
| | SEQ ID NO: 47 |
| 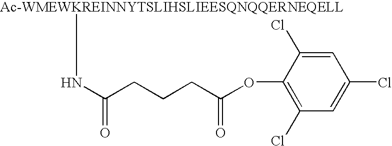 | |
| | SEQ ID NO: 48 |
| 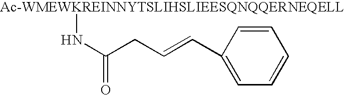 | |
| | SEQ ID NO: 49 |
| 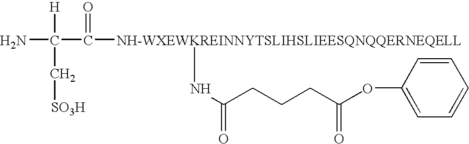 | |
| | SEQ ID NO: 50 |
| 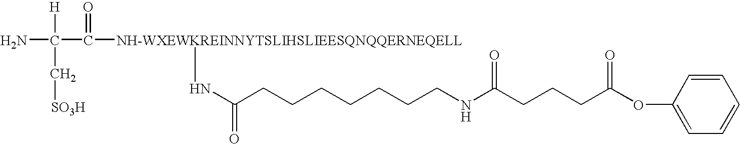 | |
| | SEQ ID NO: 51 |
| 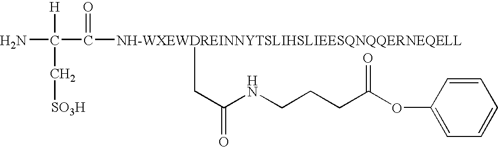 | |
| | SEQ ID NO: 52 |
| 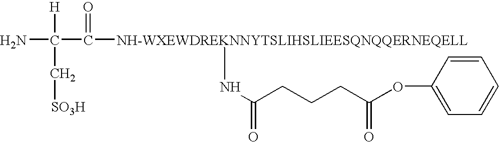 | |
| | SEQ ID NO: 53 |
| 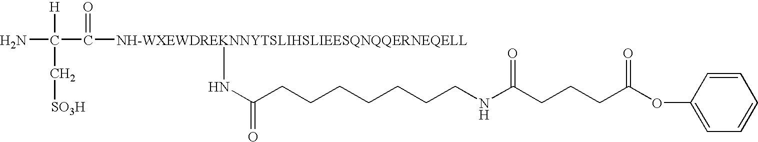 | |
| | SEQ ID NO: 54 |
| 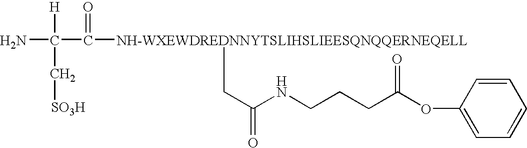 | |
| | SEQ ID NO: 55 |
| 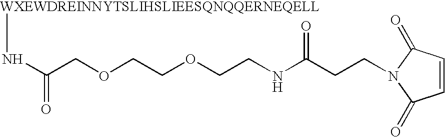 | |
| | SEQ ID NO: 56 |
| 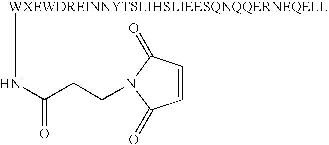 | |
| | SEQ ID NO: 57 |
| 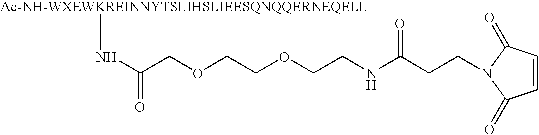 | |
| | SEQ ID NO: 58 |
| 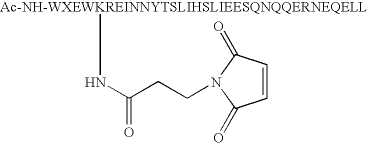 | |
| | SEQ ID NO: 59 |
| 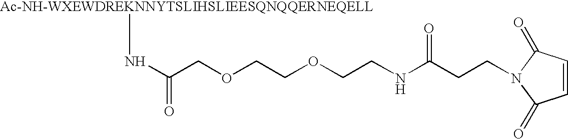 | |
| | SEQ ID NO: 59 |
| 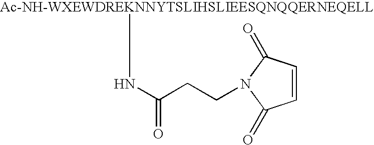 | |
| | SEQ ID NO: 60 |
| 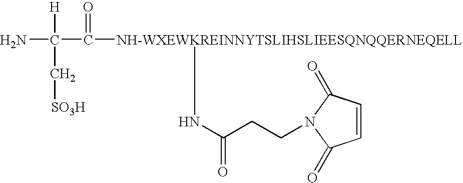 | |
| | SEQ ID NO: 61 |
| 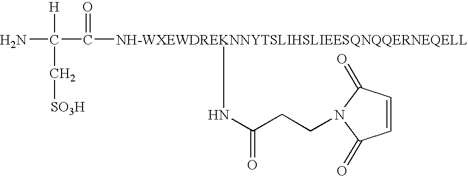 | |
| | SEQ ID NO: 62 |
| 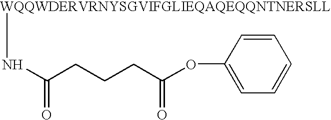 | |
| | SEQ ID NO: 63 |
| 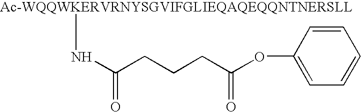 | |
| | SEQ ID NO: 64 |
| 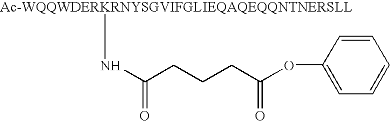 | |
| | SEQ ID NO: 65 |
| 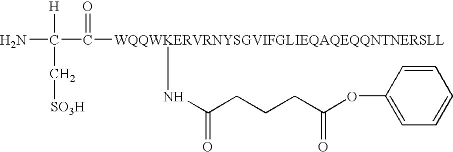 | |
| | SEQ ID NO: 66 |
| 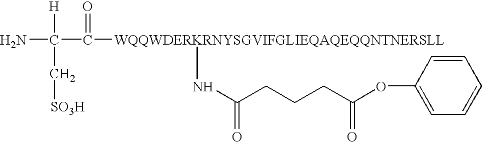 | |
| | SEQ ID NO: 67 |
| 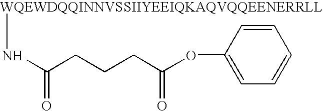 | |
| | SEQ ID NO: 68 |
| 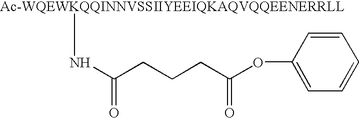 | |
| | SEQ ID NO: 69 |
| 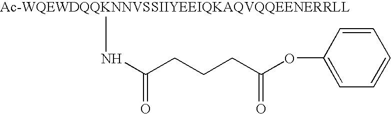 | |
| | SEQ ID NO: 70 |
| 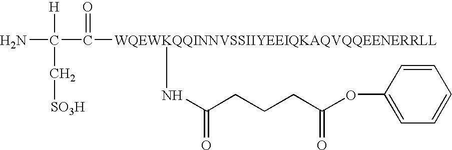 | |
| | SEQ ID NO: 71 |
| 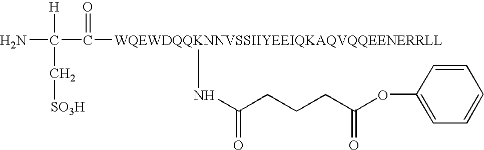 | |
| | SEQ ID NO: 72 |
| 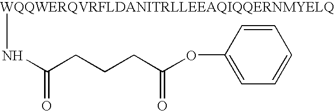 | |
| | SEQ ID NO: 73 |
| 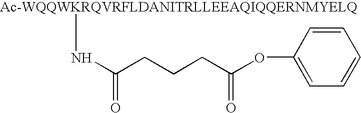 | |
| | SEQ ID NO: 74 |
| 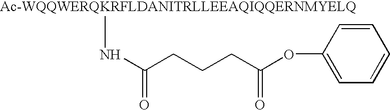 | |
| | SEQ ID NO: 75 |
| 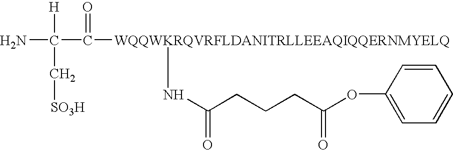 | |
| | SEQ ID NO: 76 |
| 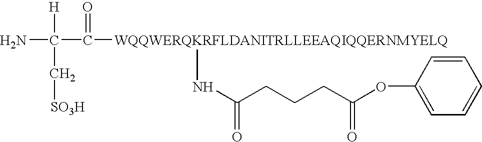 | |
| | SEQ ID NO: 77 |
| 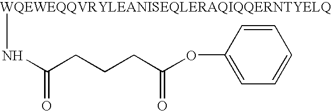 | |
| | SEQ ID NO: 78 |
| 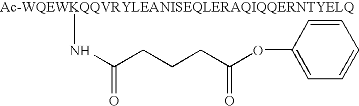 | |
| | SEQ ID NO: 79 |
| 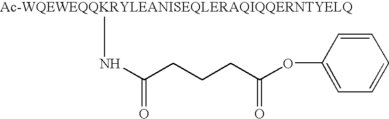 | |
| | SEQ ID NO: 80 |
| 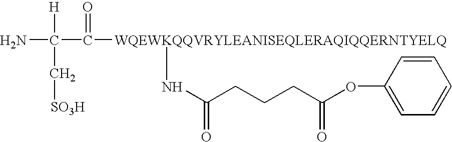 | |
| | SEQ ID NO: 81 |
| 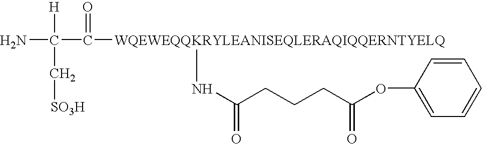 | |
| | SEQ ID NO: 82 |
| 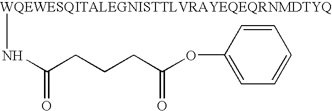 | |
| | SEQ ID NO: 83 |
| 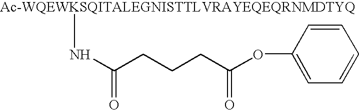 | |
| | SEQ ID NO: 84 |
| 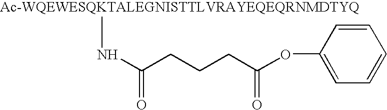 | |
| | SEQ ID NO: 85 |
| 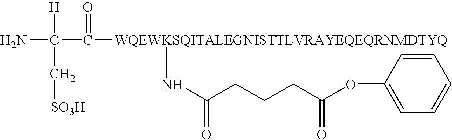 | |
| | SEQ ID NO: 86 |
| 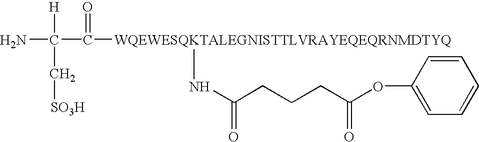 | |

TABLE 3
Example of synthetized compounds

| | Molecular weight |
|---|---|
| SEQ ID NO: | (Dalton) |
| 29 | 4664.1 |
| 31 | 4681.8 |
| 32 | 4678.8 |
| 33 | 4698.0 |
| 34 | 4501.8 |
| 35 | 4679.7 |
| 38 | 4465.5 |
| 39 | 4765.5 |
| 40 | 4767.9 |
| 41 | 4568.7 |
| 42 | 4661.7 |
| 43 | 4520.7 |
| 44 | 4538.4 |
| 45 | 4681.8 |
| 46 | 4556.4 |
| 47 | 4623.3 |

As previously mentioned, a linker may be added between B and R1, or M and R1 depending on the compound configuration, to facilitate the reaction between the compound and the target molecule. Typically, the linkers are of about 1-20 atoms in length, which atoms may be carbon, nitrogen, oxygen, sulfur, phosphorus and the like. The linkers may be alkylene groups, generally of about 2-16 carbon atoms, more generally of about 1-25 carbon atoms; polyoxyalkylene groups, where the alkylene groups will be of 2-3 atoms, and having about 1-8 units and preferably about 1-6 units; an amino acid including alpha and omega amino acids, or oligonucleotide having about 1-8 amino acids and preferably 1-6 amino acids, where the amino acids may be polar or non-polar, charged or uncharged, aliphatic, alicyclic, aromatic or heterocyclic, naturally occurring or synthetic, dextrogyre (D) or levogyre (L). In the present invention, it is preferred that the linker has the formula —NH—(CH₂)ₙ—C(O)—, where n is an integer varying from 1 to 25, more preferably, the linker is chosen from —NH—(CH₂)₅—C(O)— and —NH—CH₂—C—(O)—. Examples of linkers suitable for this purpose are illustrated at Table 3, which is only for the purpose of illustration and should not be read as limiting the scope of what a linker is contemplated in the present application.

TABLE 4
Compounds comprising a linker

| | |
|---|---|
| | SEQ ID NO: 87 |
| 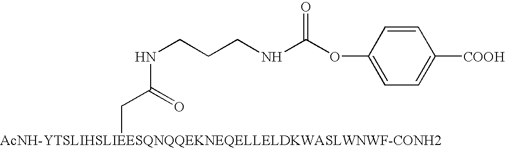 | |
| | SEQ ID NO: 88 |
| 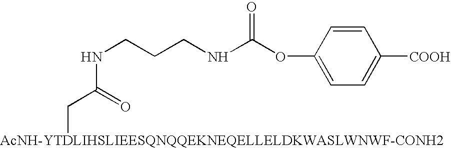 | |
| | SEQ ID NO: 89 |
| 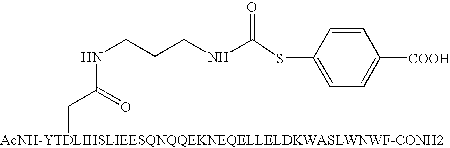 | |

It may be chosen, for design purposes, not to use an existing carboxylic group for the design of the compound. In this case, it can be appropriate to perform a deletion/addition of a new residue at a position where such mutation does not decrease/alter the binding capabilities of the binding element. An example of such transformation using SEQ ID NO:2, is illustrated below. A similar transformation could be performed anywhere on the peptide as one skilled in the art would know.

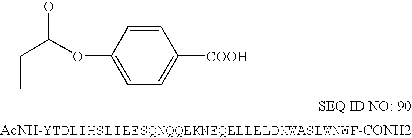

The Table 4 below illustrates compounds of the present invention wherein the binding element have a sequence selected from SEQ ID NO:3 and SEQ ID NO:4, which are other inhibitors of viral fusion.

TABLE 5

| | |
|---|---|
| | SEQ ID NO: 91 |
| 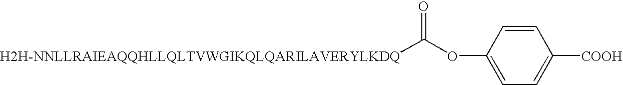 | |
| | SEQ ID NO: 92 |
| 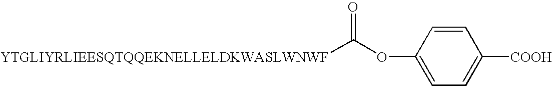 | |

Optionally, M is a moiety that comprises a therapeutic agent. Therefore, the effect of the therapeutic agent acts in addition to the deactivating action of the binding element. Preferably, the therapeutic agent is useful for anti-viral treatments, and more preferably for anti-HIV treatments, and may be, without limitation, a drug, protease inhibitor, antiproliferative agent, antisense oligonucleotide, antiviral agent, virus entry inhibitor or anti-fusiogenic agent.

### DESCRIPTION OF A SECOND PREFERRED EMBODIMENT OF THE INVENTION

According to a second preferred embodiment, the reactive group is R1-R2 and is oriented in the formula of the compound as follows: B—R2-R1-M. In this case, the binding element B is released with R2 and M stays covalently attach to the target through the bonding of R1. M is a moiety and the nature of the moiety is such that it modulates at least one specific activity of the target. The moiety preferably comprises a bulky agent selected from the group consisting of a protein (endogenous, genomic or recombinant) (i.e. recombinant serum protein), a molecule, a particle, a polymer, a liposome and a cell. For example, the moiety could be albumin.

An application of the second preferred embodiment is found to be useful for stopping, reducing or preventing viral infection. In this application, the target is a virus and the compound comprises a binding element that has a binding affinity for a region of gp41 glycoprotein or gp41 glycoprotein analog of the virus. Such binding elements are preferably the ones illustrated in Table 1 when the virus is HIV. It should be understood that the viral infection activity of other viruses such as, but not limited to, SIV, RSV, HPV (HPIV), MeV could be stopped, prevented or reduced by the compound according to the second preferred embodiment of the invention. In this case, M is preferably a moiety having the nature being such that it stops, or reduces the cell infection activity of the virus by interfering with the membrane fusion process. Preferably, the size of the moiety physically interferes with the folding of gp41 glycoprotein or its analog thereby blocking the membrane fusion process.

### EXAMPLE 1

### General Procedures for the Preparation of Cap Compounds

The peptide (20 mg) in DMF (1 mL) was reacted with the activated pentafluorophenyl (pFP) ester in the presence of 4-methylmorpholine (20 μL) for 4 h at room temperature. The reaction was quenched by addition of AcOH and then diluted with water to 20 mL. The aqueous solution was injected into semi-preparative HPLC (Phenomenex luna, RP-18, 10μ phenyl-hexyl 250×21.2 mm column, flow rate 9.5 mL/min with collection of 9.5 mL fractions. A gradient of 30 to 60% acetonitrile (0.1% TFA) in water (0.1% TFA) over 120 min was used) to give corresponding cap peptide. The pure fractions were combined and lyophilized to give a white powder.

Linker Synthesis

The substituted phenol was reacted with glutaric anhydride (1.5 equivalent) in the presence of Et3N (2 equivalents) in dichloromethane. The corresponding acid was isolated by flash column chromatography to give the phenol ester of the glutaric acid.

The acid was activated by the reaction of pentafluorophenol (2.2 equivalents) and EDC (2.2 equivalents) in dichloromethane for 16 h. The activated pentafluorophenyl ester was purified by flash column chromatography to give a solid or an oil.

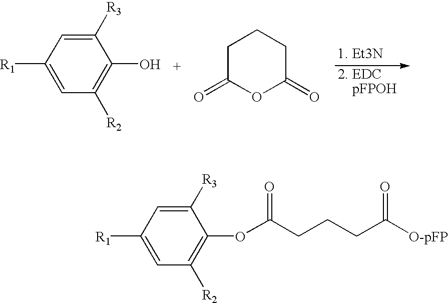

The long linker version was made by the coupling the activated esters produced above with 8-aminooctanoic acid in DMF in the presence of NMM. The acid was reacted with pentafluorophenol and EDC to give the corresponding pFP ester after flash chromatography.

### EXAMPLE 2

### Determination of IC-50 for Assessing the Activity of Antiviral Drugs Using HIV-1 Strain IIIB

Reagents

The assay is performed in a 96-well plate with 500,000 PHA-stimulated CBMC (cord blood mononuclear cells) per well, 2 wells per drug concentration, with a minimum of 6 concentrations per drug. Media used is RPMI 1640 with 10% FBS, penicilin (100 U/ml), streptomycine (100 μg/ml), glutamine (2 mM), hydrocortisone (5 μg/ml), and IL-2 (20 U/ml).

Drug Preparation

Control drug stocks are prepared by adding 2 ml of buffer to the native compounds or the reactive compounds. The stocks are stored at −20 degrees Celsius. The drug concentrations were determined by ConjuChem Inc. 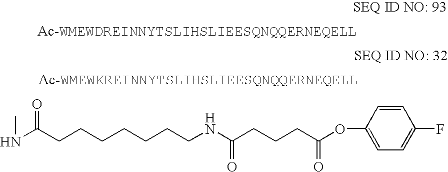
Addition of the Compound Prior to Cell Infection

Target cells are incubated with different compounds concentrations for 30-60 minutes at 37 degrees Celsius prior to addition of virus (multiplicity of infection (MOI)=0.1-1.0). Following plating, target cells are in the continuous presence of the different concentrations of inhibitor for a total of 7 days.

Following 3-4 days of incubation, each compound is replenished.

Reverse transcriptase assay is used to measure the IC50.

CC50 (cytotoxic concentration) is also evaluated for all compounds.

3TC is used as a control inhibitor

RT Results (cpm) (Day 7):

A. +drugs 7 days

| Conc (nm) | SEQ ID NO: 93 | | | SEQ ID NO: 32 | | |
|---|---|---|---|---|---|---|
| 0 | 48951 | 46318 | 47432 | 71900 | 54074 | 50994 |
| 125 | 5854 | 5040 | 5219 | 6322 | 6929 | 5909 |
| 600 | 4924 | 5850 | 5093 | 6477 | 11889 | 6030 |
| 3000 | 5811 | 6955 | 5973 | 7967 | 5896 | 6208 |

Following the chemical insertion of (linker-R1-R2-M) within the peptide sequence of C34 (at position Asp5) in SEQ ID NO:93, there is essentially no loss in anti-HIV activity observed as compared to a native (non-covalent) version of C34 (SEQ ID NO:32). Hence, the binding element (B) found within SEQ ID NO:32 is as efficient in binding to its viral target as native C34 (SEQ ID NO:93) despite the chemical insertions on an amino-acid residue known to be in direct contact with the viral target (i.e. The N-heptad repeat of HIV-1's gp41).

HPLC
CJC-1722 or SEQ ID NO:94:
Ac-SGIVQQQNNLLRAIEAQQHLLQLTVWGIKQLQARIL-CONH₂
CJC-1723 or SEQ ID NO:95:
Ac-SGIVQQQNNLLRAIEAQQHLLQLTVWGIRQLQARIL-CONH₂
CJC-1592 or SEQ ID NO:96:
SGIVQQQNNLLRAIEAQQHLLQLTVWGIKQLQARIL-CONH₂

Reaction in Phosphate Buffer
2 mM of each C34 prepared in 50 mM sodium phosphate buffer (pH 7)
10 mM Native N36 prepared in 95% ETOH→diluted to 2 mM in 50 mM Tris (pH 10.7)+19% ETOH
0.2 mM DAC:C34+0.2 mM N36 in 50 mM sodium phosphate buffer (pH 7)+1.9% ETOH @ 37° C.
Inject 10 μl of peptide solution into HPLC or LC/MS
   Reaction in 50/50 Phosphate Buffer+EtOH
2 mM of each C34 prepared in 50 mM sodium phosphate buffer (pH 7)
Native N36 prepared at 2 mM in 95% ETOH
0.2 mM C34+0.2 mM N36 in 50% EtOH/50% sodium phosphate buffer (pH 7) @ 37° C.
Inject 10 μl of peptide solution into HPLC or LC/MS

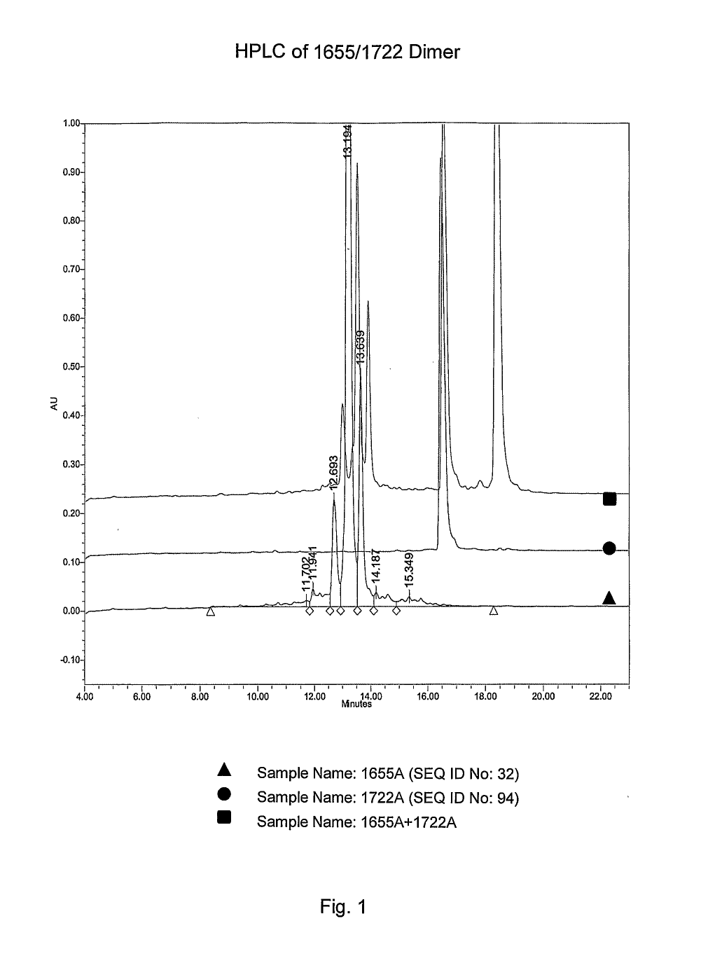

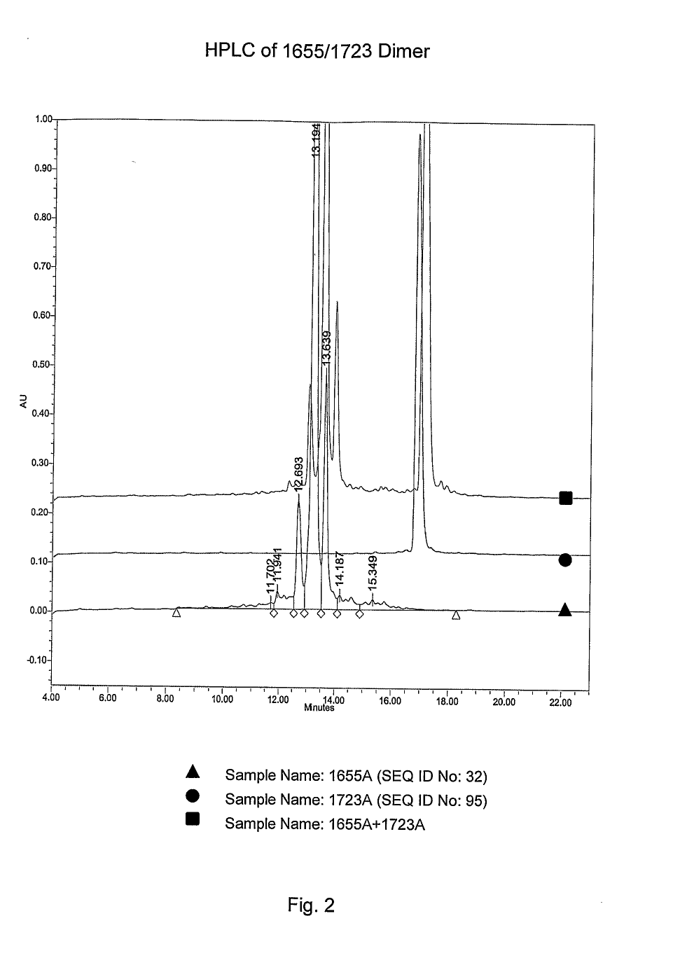

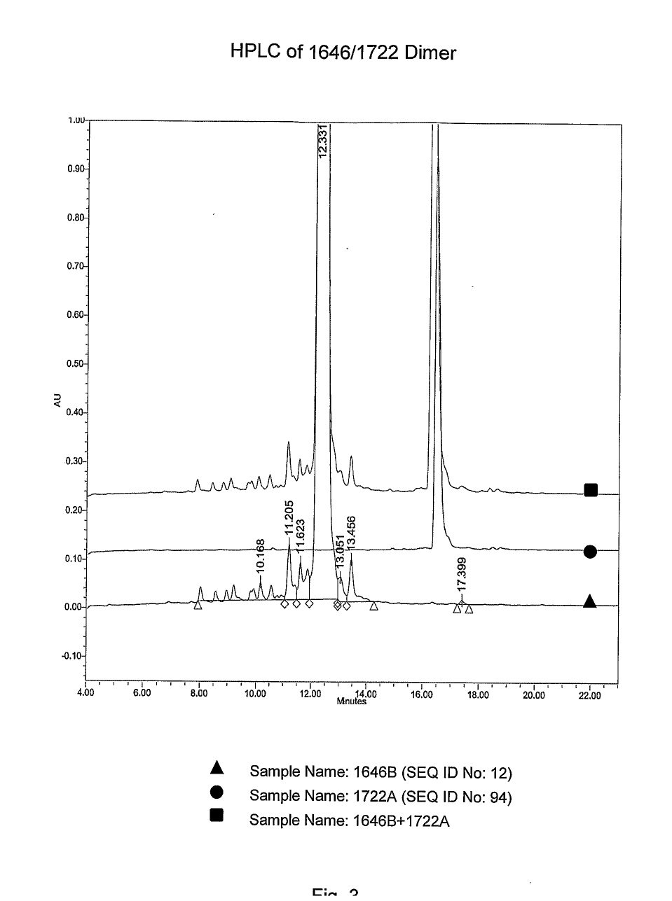

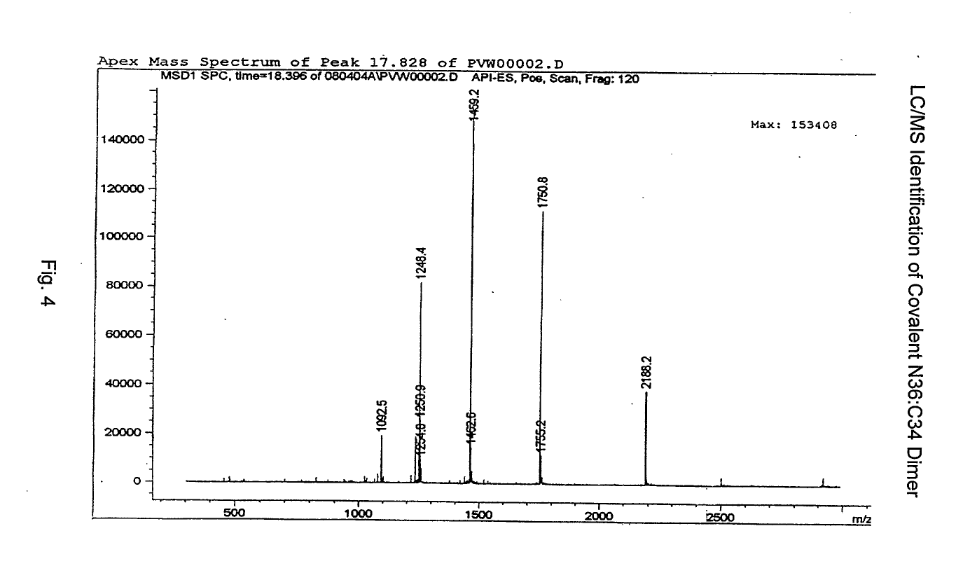

Results are presented in FIGS. 1 to 4. In addition to not perturbing binding affinity, the insertion of a reactive moiety at position 5 of C34 (i.e. aspartic acid at position 5) forms a covalent bond specifically the sole Lysine residue of the N36 peptide.

Page

Following the 1 hour incubation at room temperature, at least 20 micrograms of total N36/C34 peptide mixture were loaded into each well and separated using Native-polyacrylamide gel electrophoresis (17.5% acrylamide) and stained using Coomassie Brilliant Blue. Unlike SDS-PAGE, Native-PAGE allows one to visualize the formation of six-helix bundles at approximately 28 kDa composed of three central N36 peptides surrounded by three C34 peptides. The formation of this structure and its analogs among other viruses is critical to the subsequent membrane fusion events.

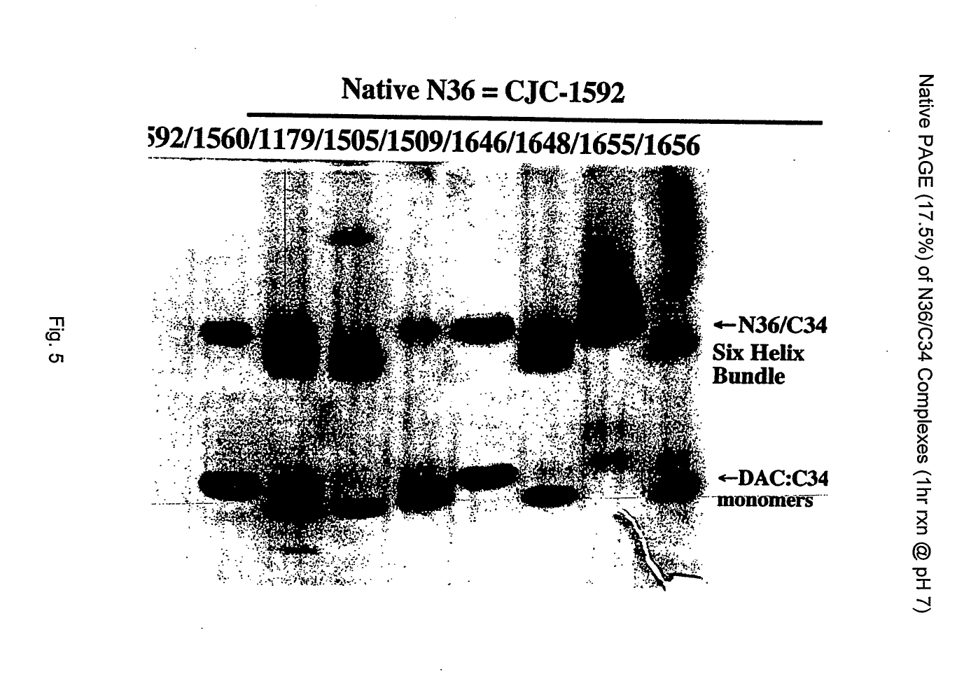

Results are shown in FIG. 5.

In addition to the ability of SEQ ID NO:32 (CJC-1655) to inhibit the reverse-transcriptase (RT) activity of HIV-1 using a standard in vitro assay as effectively as the native C34 (CJC-1646, SEQ ID NO:93), Native-PAGE indicates that the insertion of a linker and R1 to a wide variety of positions within the structure of C34 (i.e. N-terminus (CJC-1505 and CJC-1648, SEQ ID NO:55); D5 (CJC-1655, SEQ ID NO:32); 18 (CJC-1656, SEQ ID NO:59); N9 (CJC-1 179, C34 with N9 Lys(AEEA)-MPA) and K35 (CJC-1509)) does not impede C34 from binding to its target N36 peptide (CJC-1592) derived from the N-heptad repeat of HIV-1's gp41 glycoprotein, as compared to the native (unreactive) versions of C34 (CJC-1560, SEQ ID NO:5 and CJC-1646, SEQ ID NO:93).

While the invention has been described in connection with specific embodiments thereof, it will be understood that it is capable of further modifications, and this application is intended to cover any variations, uses or adaptations of the invention following, in general, the principles of the invention, and including such departures from the present description as come within known or customary practice within the art to which the invention pertains, and as may be applied to the essential features hereinbefore set forth, and as follows in the scope of the appended claims.

What is claimed is:

1. An antiviral compound having a formula I:
B—R1-R2-M I
wherein:
B is a binding element for recognizing and binding a viral target;
R1 is a first group of atoms for reacting with a functionality of said viral target so as to form a covalent bond with said target;
R2 is a second group of atoms; R1 and R2 being such that formation of the covalent bond between R1 and said target generates cleavage of the bond between R1 and R2 so as to free R2-M; and
M is selected from the group consisting of a hydrogen atom and a pharmaceutically acceptable moiety;
or
a formula II:
B—R2-R1-M II
wherein:
B is a binding element for recognizing and binding a viral target;
R1 is a first group of atoms for reacting with a functionality of said target so as to form a covalent bond with said target;
R2 is second group of atoms; R1 and R2 being such that formation of the covalent bond between R1 and said target generates cleavage of the bond between R1 and R2 so as to free B—R2; and
M is a pharmaceutically acceptable moiety.

2. The antiviral compound as claimed in claim 1, wherein R1 is selected from the group having a formula III:
X—C(Y) III
wherein:
X is absent or selected from the group consisting of alkyl groups, and substituted or unsubstituted phenyl groups; and
Y is selected from the group consisting of a hydrogen atom, a sulfur atom, an oxygen atom and a nitrogen atom.

3. The antiviral compound as claimed in claim 2, wherein said substituted phenyl group bears at least one substituent selected from the group consisting of a halogen, NO₂, SO₂NH₂, SO₂NHF, CF₃, CCl₃, CBr₃, C═N, SO₃H, CO₂H, CHO, NHR, OH, NHCOCH₃, OCH₃, CH₃ and CH₂CH₃.

4. The antiviral compound as claimed in claim 1, wherein R1 is selected from the group consisting of sulfur, oxygen, phosphorus and nitrogen.

5. The antiviral compound as claimed in claim 2, wherein Y is selected from the group consisting of sulfur and oxygen.

6. The antiviral compound as claimed in anyone of claim 1 to 5, wherein R2 is selected from the group consisting of oxygen, acetal, hemiacetal, phosphoacetal, sulfur, alkoxy, thioalkoxy, hydroxyamino derivatives, substituted or unsubstituted phenoxy, substituted or unsubstituted thiophenoxy, and substituted or unsubstituted aminophenoxy derivatives.

7. The antiviral compound as claimed in claim 6, wherein the substituted phenoxy, thiophenoxy, or aminophenoxy derivative contains a substituted phenyl group which bears at least one substituent selected from the group consisting of a halogen, NO₂, SO₂NH₂, SO₂NHF, CF₃, CCl₃, CBr₃, C═N, SO₃H, CO₂H, CHO, NHR, OH, NHCOCH₃, OCH₃, CH₃ and CH₂CH₃.

8. The antiviral compound as claimed in claim 1, wherein R1-R2 comprises a functional group selected from alkyl esters, aryl esters, alkyl thioesters, aryl thioesters, phosphoesters, ortho esters, imidates, mixed anhydrides, disulphides, amides and thioamines.

9. The antiviral compound as claimed in claim 1, wherein R1 is a carbonyl group and R2 is an oxygen atom.

10. The antiviral compound as claimed in any one of claims 1-9 wherein said functionality of the viral target is selected from the group consisting of NH₂.

11. The antiviral compound as claimed in any one of claims 1-10, wherein said binding element is selected from the group consisting of an organic compound, an amino acid sequence, a peptide, a protein, a nucleic acid sequence, a small molecule, a mimetic thereof and a combination thereof.

12. The antiviral compound as claimed in claim 1, wherein said viral target is selected from the group consisting of a virus, a viral antigen expressed on the surface of an infected cell, a ligand specific to a virus or a viral antigen of a surface receptor on an infected cell, an infected cell surface receptor, a peptide, an infected cell or membrane thereof, a viral protein expressed at the surface of an infected cell, fragments thereof or specific regions thereof.

13. The antiviral compound as claimed in claim 12, wherein said viral protein is gp41.

14. The antiviral compound as claimed in claim 12, wherein said specific regions are N-heptad repeat and C-heptad repeat of gp-41.

15. The antiviral compound as claimed in claim 1, wherein said viral target is a virus.

16. The antiviral compound as claimed in claim 15, wherein said virus is selected from the group consisting of Human Immunodeficiency Virus (HIV-1 and 2), Respiratory Syncytial virus (RSV), influenza virus, human Papilloma Virus (HPV), Ebola, dengue, rubella, Epstein Barr, Hepatitis, HTLV-1 and 2, Semliki Forest Virus (SFV), Measle Virus (MeV), yellow fever, Japanese encephalitis, West Nile and tick-borne encephalitis (TBE) viruses.

17. The antiviral compound as claimed in claim 16, wherein said binding element has a binding affinity for a region of gp41 glycoprotein or analog thereof of said virus.

18. The antiviral compound as claimed in claim 17, wherein the binding element has an amino acid sequence selected from the group consisting of:
(SEQ ID NO:1)
YTSLIHSLIEESQNQQEKNEQELLELDKWASLWNWF;
(SEQ ID NO:2)
YTDLIHSLIEESQNQQEKNEQELLELDKWASLWNWF;
(SEQ ID NO:3)
NNLLRAIEAQQHLLQLTVWQIKQLQARILAVERYLKDQ;
(SEQ ID NO:4)
YTGLIYRLIEESQTQQEKNELLELDKWASLWNWF;
(SEQ ID NO:5)
WMEWDREINNYTSLIHSLIEESQNQQEKNEQELL;
(SEQ ID NO:6)
WXEWDRKIEEYTKKIKKLIEESQEQQEKNEKELK;
(SEQ ID NO:7)
WXEWDRKIEEYTKKIEELIKKSQEQQEKNEKELK;
(SEQ ID NO:8)
WEEWDKKIEEYTKKIEELIKKSEEQQKKNEEELKK;
(SEQ ID NO:9)
WMEWDREINNYTSLIHSLIEESQNQQEKNEQELLEL;
(SEQ ID NO:10)
WMEWDREINNYTSLIHSLIEESQNQQEKNEQELLK;
(SEQ ID NO:11)
WMEWDREIKNYTSLIHSLIEESQNQQEKNEQELL;
(SEQ ID NO:12)
WMEWDREIEINNYTSLIHSLIEESQNQQERNEQELL;
(SEQ ID NO:13)
WMEWKREINNYTSLIHSLIEESQNQQERNEQELL;
(SEQ ID NO:14)
WMEWDREKNNYTSLIHSLIEESQNQQERNEQELL;
(SEQ ID NO:15)
WQQWDEKVRNYSGVIFGLIEQAQEQQNTNEKSLL;
(SEQ ID NO:16)
WQEWDQQINNVSSIIYEEIQKAQVQQEENEKKLL;
(SEQ ID NO:17)
WQQWERQVRFLDANITKLLEEAQIQQEKNMYELQ;
(SEQ ID NO:18)
WQEWEQQVRYLEANISEQLERAQIQQEKNTYELQ;
and
(SEQ ID NO:19)
WQEWESQITALEGNISTTLVKAYEQEQKNMDTYQ.

19. The antiviral compound as claimed in claim 18, which further comprises one acetyl or at least one cysteic acid at the N-terminus of said amino acid sequence.

20. The antiviral compound as claimed in anyone of claims 1 to 19, wherein M comprises a therapeutic agent.

21. The antiviral compound as claimed in claim 20, wherein said therapeutic agent is selected from the group consisting of drugs, antisense oligonucleotides, antiviral agents, virus entry inhibitors and anti-fusiogenic agents.

22. The antiviral compound as claimed in claim 1, further comprising a linker L1 between B and R1.

23. The antiviral compound as claimed in claim 1 or 22, further comprising a linker L2 between R2 and M.

24. The antiviral compound as claimed in claim 22 or 23, wherein said linker is a group of 1-20 atoms, said atoms being selected from the group consisting of carbon, nitrogen, oxygen, sulfur and phosphorus.

25. The antiviral compound as claimed in claim 22 or 23, wherein said linker is selected from the group consisting of an alkylene group, a polyoxyalkylene group, an amino acid and an oligopeptide.

26. The antiviral compound as claimed in claim 25, wherein said alkylene group comprises 1 to 25 carbon atoms.

27. The antiviral compound as claimed in claim 25, wherein said alkylene group comprises 2 to 16 carbon atoms.

28. The antiviral compound as claimed in claim 25, wherein said polyoxyalkylene group comprises alkylene groups of 2 to 3 carbon atoms and comprises 1 to 8 units.

29. The antiviral compound as claimed in claim 25, wherein said polyoxyalkylene group comprises alkylene groups of 2 to 3 carbon atoms and comprises 1 to 6 units.

30. The antiviral compound as claimed in claim 25, wherein said oligopeptide comprises 1 to 8 amino acids.

31. The antiviral compound as claimed in claim 25, wherein said oligopeptide comprises 1 to 6 amino acids.

32. The antiviral compound as claimed in claim 25, wherein said linker has the formula —NH—(CH₂)ₙ—C(O)—, where n is an integer varying from 1 to 25.

33. The antiviral compound as claimed in claim 25, wherein said linker is —NH—(CH₂)₅—C(O)—.

34. The antiviral compound as claimed in claim 25, wherein said linker is —NH—CH₂—C(O)—.

35. An antiviral compound selected from the group consisting of SEQ ID NOs:20 to 92.

36. An antiviral composition for modulating an activity of a viral target comprising a compound as claimed in anyone of claims 1 to 35 in association with a pharmaceutically acceptable carrier; said binding element having a binding affinity for a region of the viral target involved in the activity of a membrane fusion process of cell infection of a virus, whereby the bonding of said compound to said region of the target results in the interruption or reduction of the activity of the target.

37. A method for modulating an activity of a viral target in a subject comprising administering to said subject the compound as claimed in anyone of claims 1 to 35, alone or in association with a pharmaceutically acceptable carrier; said binding element having a binding affinity for a region of the viral target involved in the activity of a membrane fusion process of cell infection of a virus, whereby the bonding of said compound to said region of the target results in the interruption or reduction of the activity of the target.

38. Use of a compound for the manufacture of a medicament for use in an antiviral treatment of a subject, the compound being as claimed in anyone of claims 1 to 35.

39. Use of claim 38, wherein the subject is infected by a virus selected from the group consisting of Human Immunodeficiency Virus (HIV-1 and 2), Respiratory Syncytial virus (RSV), influenza virus, human Papilloma Virus (HPV), Ebola, dengue, rubella, Epstein Barr, Hepatitis, HTLV-1 and 2, Semliki Forest Virus (SFV), Measle Virus (MeV), yellow fever, Japanese encephalitis, West Nile and tick-borne encephalitis (TBE) viruses.


SEQUENCE LISTING (96 sequences)

SEQ ID NO: 1 - protein, 36 aa, Artificial Sequence
  Synthetically generated peptide
YTSLIHSLIEESQNQQEKNEQELLELDKWASLWNWF

SEQ ID NO: 2 - protein, 36 aa, Artificial Sequence
  Synthetically generated peptide
YTDLIHSLIEESQNQQEKNEQELLELDKWASLWNWF

SEQ ID NO: 3 - protein, 38 aa, Artificial Sequence
  Synthetically generated peptide
NNLLRAIEAQQHLLQLTVWGIKQLQARILAVERYLKDQ

SEQ ID NO: 4 - protein, 34 aa, Artificial Sequence
  Synthetically generated peptide
YTGLIYRLIEESQTQQEKNELLELDKWASLWNWF

SEQ ID NO: 5 - protein, 34 aa, Artificial Sequence
  Synthetically generated peptide
WMEWDREINNYTSLIHSLIEESQNQQEKNEQELL

SEQ ID NO: 6 - protein, 34 aa, Artificial Sequence
  Synthetically generated peptide
  misc_feature 2: Xaa = any naturally occurring amino acid
WXEWDRKIEEYTKKIKKLIEESQEQQEKNEKELK

SEQ ID NO: 7 - protein, 34 aa, Artificial Sequence
  Synthetically generated peptide
  misc_feature 2: Xaa = any naturally occurring amino acid
WXEWDRKIEEYTKKIEELIKKSQEQQEKNEKELK

SEQ ID NO: 8 - protein, 35 aa, Artificial Sequence
  Synthetically generated peptide
WEEWDKKIEEYTKKIEELIKKSEEQQKKNEEELKK

SEQ ID NO: 9 - protein, 36 aa, Artificial Sequence
  Synthetically generated peptide
WMEWDREINNYTSLIHSLIEESQNQQEKNEQELLEL

SEQ ID NO: 10 - protein, 35 aa, Artificial Sequence
  Synthetically generated peptide
WMEWDREINNYTSLIHSLIEESQNQQEKNEQELLK

SEQ ID NO: 11 - protein, 34 aa, Artificial Sequence
  Synthetically generated peptide
WMEWDREIKNYTSLIHSLIEESQNQQEKNEQELL

SEQ ID NO: 12 - protein, 34 aa, Artificial Sequence
  Synthetically generated peptide
WMEWDREINNYTSLIHSLIEESQNQQERNEQELL

SEQ ID NO: 13 - protein, 34 aa, Artificial Sequence
  Synthetically generated peptide
WMEWKREINNYTSLIHSLIEESQNQQERNEQELL

SEQ ID NO: 14 - protein, 34 aa, Artificial Sequence
  Synthetically generated peptide
WMEWDREKNNYTSLIHSLIEESQNQQERNEQELL

SEQ ID NO: 15 - protein, 34 aa, Artificial Sequence
  Synthetically generated peptide
WQQWDEKVRNYSGVIFGLIEQAQEQQNTNEKSLL

SEQ ID NO: 16 - protein, 34 aa, Artificial Sequence
  Synthetically generated peptide
WQEWDQQINNVSSIIYEEIQKAQVQQEENEKKLL

SEQ ID NO: 17 - protein, 34 aa, Artificial Sequence
  Synthetically generated peptide
WQQWERQVRFLDANITKLLEEAQIQQEKNMYELQ

SEQ ID NO: 18 - protein, 34 aa, Artificial Sequence
  Synthetically generated peptide
WQEWEQQVRYLEANISEQLERAQIQQEKNTYELQ

SEQ ID NO: 19 - protein, 34 aa, Artificial Sequence
  Synthetically generated peptide
WQEWESQITALEGNISTTLVKAYEQEQKNMDTYQ

SEQ ID NO: 20 - protein, 36 aa, Artificial Sequence; identical to SEQ ID NO: 1
  Synthetically generated peptide
  ACETYLATION 1
  BINDING (36)..(36): organic compound

SEQ ID NO: 21 - protein, 36 aa, Artificial Sequence; identical to SEQ ID NO: 1
  Synthetically generated peptide
  ACETYLATION 1
  BINDING (26)..(26): organic compound

SEQ ID NO: 22 - protein, 36 aa, Artificial Sequence; identical to SEQ ID NO: 1
  Synthetically generated peptide
  ACETYLATION 1
  BINDING (25)..(25): organic compound

SEQ ID NO: 23 - protein, 36 aa, Artificial Sequence; identical to SEQ ID NO: 1
  Synthetically generated peptide
  ACETYLATION 1
  BINDING (22)..(22): organic compound compound

SEQ ID NO: 24 - protein, 36 aa, Artificial Sequence
  Synthetically generated peptide
  ACETYLATION 1
  BINDING (20)..(20): organic compound
YTSLIHSLIEESQNQQEKNEQELLELDKWASLWMWF

SEQ ID NO: 25 - protein, 36 aa, Artificial Sequence; identical to SEQ ID NO: 1
  Synthetically generated peptide
  ACETYLATION 1
  BINDING (17)..(17): organic compound

SEQ ID NO: 26 - protein, 36 aa, Artificial Sequence; identical to SEQ ID NO: 1
  Synthetically generated peptide
  ACETYLATION 1
  BINDING (11)..(11): organic compound

SEQ ID NO: 27 - protein, 36 aa, Artificial Sequence; identical to SEQ ID NO: 1
  Synthetically generated peptide
  ACETYLATION 1
  BINDING 10: organic compound

SEQ ID NO: 28 - protein, 36 aa, Artificial Sequence; identical to SEQ ID NO: 1
  Synthetically generated peptide
  ACETYLATION 1
  BINDING 10: organic compound

SEQ ID NO: 29 - protein, 34 aa, Artificial Sequence; identical to SEQ ID NO: 14
  Synthetically generated peptide
  ACETYLATION 1
  BINDING 8: organic compound

SEQ ID NO: 30 - protein, 34 aa, Artificial Sequence; identical to SEQ ID NO: 14
  Synthetically generated peptide
  ACETYLATION 1
  BINDING 8: organic compound

SEQ ID NO: 31 - protein, 34 aa, Artificial Sequence; identical to SEQ ID NO: 14
  Synthetically generated peptide
  ACETYLATION 1
  BINDING 8: organic compound

SEQ ID NO: 32 - protein, 34 aa, Artificial Sequence; identical to SEQ ID NO: 13
  Synthetically generated peptide
  ACETYLATION 1
  BINDING 5: organic compound

SEQ ID NO: 33 - protein, 34 aa, Artificial Sequence; identical to SEQ ID NO: 13
  Synthetically generated peptide
  ACETYLATION 1
  BINDING 5: organic compound

SEQ ID NO: 34 - protein, 34 aa, Artificial Sequence; identical to SEQ ID NO: 12
  Synthetically generated peptide
  BINDING 1: organic compound

SEQ ID NO: 35 - protein, 34 aa, Artificial Sequence; identical to SEQ ID NO: 12
  Synthetically generated peptide
  BINDING 1: organic compound

SEQ ID NO: 36 - protein, 34 aa, Artificial Sequence; identical to SEQ ID NO: 13
  Synthetically generated peptide
  ACETYLATION 1
  BINDING 5: organic compound

SEQ ID NO: 37 - protein, 34 aa, Artificial Sequence; identical to SEQ ID NO: 12
  Synthetically generated peptide
  BINDING 1: organic compound

SEQ ID NO: 38 - protein, 34 aa, Artificial Sequence; identical to SEQ ID NO: 12
  Synthetically generated peptide
  BINDING 1: organic compound

SEQ ID NO: 39 - protein, 34 aa, Artificial Sequence; identical to SEQ ID NO: 13
  Synthetically generated peptide
  ACETYLATION 1
  BINDING 5: organic compound

SEQ ID NO: 40 - protein, 34 aa, Artificial Sequence; identical to SEQ ID NO: 14
  Synthetically generated peptide
  ACETYLATION 1
  BINDING 8: organic compound

SEQ ID NO: 41 - protein, 34 aa, Artificial Sequence; identical to SEQ ID NO: 12
  Synthetically generated peptide
  BINDING 1: organic compound

SEQ ID NO: 42 - protein, 34 aa, Artificial Sequence; identical to SEQ ID NO: 13
  Synthetically generated peptide
  ACETYLATION 1
  BINDING 5: organic compound

SEQ ID NO: 43 - protein, 34 aa, Artificial Sequence; identical to SEQ ID NO: 13
  Synthetically generated peptide
  ACETYLATION 1
  BINDING 5: organic compound

SEQ ID NO: 44 - protein, 34 aa, Artificial Sequence; identical to SEQ ID NO: 13
  Synthetically generated peptide
  ACETYLATION 1
  BINDING 5: organic compound

SEQ ID NO: 45 - protein, 34 aa, Artificial Sequence; identical to SEQ ID NO: 14
  Synthetically generated peptide
  ACETYLATION 1
  BINDING 8: organic compound

SEQ ID NO: 46 - protein, 34 aa, Artificial Sequence; identical to SEQ ID NO: 13
  Synthetically generated peptide
  ACETYLATION 1
  BINDING 5: organic compound

SEQ ID NO: 47 - protein, 34 aa, Artificial Sequence; identical to SEQ ID NO: 13
  Synthetically generated peptide
  ACETYLATION 1
  BINDING 5: organic compound

SEQ ID NO: 48 - protein, 34 aa, Artificial Sequence; identical to SEQ ID NO: 13
  Synthetically generated peptide
  ACETYLATION 1
  BINDING 5: organic compound

SEQ ID NO: 49 - protein, 34 aa, Artificial Sequence
  Synthetically generated peptide
  BINDING 1: cysteic acid
  misc_feature 2: Xaa can be any naturally occurring amino acid
  BINDING 5: organic compound
WXEWKREINNYTSLIHSLIEESQNQQERNEQELL

SEQ ID NO: 50 - protein, 34 aa, Artificial Sequence
  Synthetically generated peptide
  BINDING 1: cysteic acid
  misc_feature 2: Xaa = any naturally occurring amino acid
  BINDING 5: organic compound
WXEWDREINNYTSLIHSLIEESQNQQERNEQELL

SEQ ID NO: 51 - protein, 34 aa, Artificial Sequence; identical to SEQ ID NO: 50
  Synthetically generated peptide
  BINDING 1: cysteic acid
  misc_feature 2: Xaa = any naturally occurring amino acid
  BINDING 5: organic compound

SEQ ID NO: 52 - protein, 34 aa, Artificial Sequence
  Synthetically generated peptide
  BINDING 1: cysteic acid
  misc_feature 2: Xaa = any naturally occurring amino acid
  BINDING 8: organic compound
WXEWDREKNNYTSLIHSLIEESQNQQERNEQELL

SEQ ID NO: 53 - protein, 34 aa, Artificial Sequence; identical to SEQ ID NO: 52
  Synthetically generated peptide
  BINDING 1: cysteic acid
  misc_feature 2: Xaa can be any naturally occurring amino acid
  BINDING 8: organic compound

SEQ ID NO: 54 - protein, 34 aa, Artificial Sequence
  Synthetically generated peptide
  BINDING 1: cysteic acid
  misc_feature 2: Xaa = any naturally occurring amino acid
  BINDING 8: organic compound
WXEWDREDNNYTSLIHSLIEESQNQQERNEQELL

SEQ ID NO: 55 - protein, 34 aa, Artificial Sequence; identical to SEQ ID NO: 50
  Synthetically generated peptide
  BINDING 1: organic compound
  misc_feature 2: Xaa = any naturally occurring amino acid

SEQ ID NO: 56 - protein, 34 aa, Artificial Sequence; identical to SEQ ID NO: 50
  Synthetically generated peptide
  BINDING 1: organic compound
  misc_feature 2: Xaa = any naturally occurring amino acid

SEQ ID NO: 57 - protein, 34 aa, Artificial Sequence; identical to SEQ ID NO: 49
  Synthetically generated peptide
  ACETYLATION 1
  misc_feature 2: Xaa = any naturally occurring amino acid
  BINDING 5: organic compound

SEQ ID NO: 58 - protein, 34 aa, Artificial Sequence; identical to SEQ ID NO: 49
  Synthetically generated peptide
  ACETYLATION 1
  misc_feature 2: Xaa can be any naturally occurring amino acid
  BINDING 5: organic compound

SEQ ID NO: 59 - protein, 34 aa, Artificial Sequence; identical to SEQ ID NO: 52
  Synthetically generated peptide
  ACETYLATION 1
  misc_feature 2: Xaa = any naturally occurring amino acid
  BINDING 8: organic compound

SEQ ID NO: 60 - protein, 34 aa, Artificial Sequence; identical to SEQ ID NO: 49
  Synthetically generated peptide
  BINDING 1: cysteic acid
  misc_feature 2: Xaa = any naturally occurring amino acid
  BINDING 5: organic compound

SEQ ID NO: 61 - protein, 34 aa, Artificial Sequence; identical to SEQ ID NO: 52
  Synthetically generated peptide
  BINDING 1: cysteic acid
  misc_feature 2: Xaa = any naturally occurring amino acid
  BINDING 8: organic compound

SEQ ID NO: 62 - protein, 34 aa, Artificial Sequence
  Synthetically generated peptide
  BINDING 1: organic compound
WQQWDERVRNYSGVIFGLIEQAQEQQNTNERSLL

SEQ ID NO: 63 - protein, 34 aa, Artificial Sequence
  Synthetically generated peptide
  ACETYLATION 1
  BINDING 5: organic compound
WQQWKERVRNYSGVIFGLIEQAQEQQNTNERSLL

SEQ ID NO: 64 - protein, 34 aa, Artificial Sequence
  Synthetically generated peptide
  ACETYLATION 1
  BINDING 8: organic compound
WQQWDERKRNYSGVIFGLIEQAQEQQNTNERSLL

SEQ ID NO: 65 - protein, 34 aa, Artificial Sequence; identical to SEQ ID NO: 63
  Synthetically generated peptide
  BINDING 1: cysteic acid
  BINDING 5: organic compound

SEQ ID NO: 66 - protein, 34 aa, Artificial Sequence; identical to SEQ ID NO: 64
  Synthetically generated peptide
  BINDING 1: cysteic acid
  BINDING 8: organic compound

SEQ ID NO: 67 - protein, 34 aa, Artificial Sequence
  Synthetically generated peptide
  BINDING 1: organic compound
WQEWDQQINNVSSIIYEEIQKAQVQQEENERRLL

SEQ ID NO: 68 - protein, 34 aa, Artificial Sequence
  Synthetically generated peptide
  ACETYLATION 1
  BINDING 5: organic compound
WQEWKQQINNVSSIIYEEIQKAQVQQEENERRLL

SEQ ID NO: 69 - protein, 34 aa, Artificial Sequence
  Synthetically generated peptide
  ACETYLATION 1
  BINDING 8: organic compound
WQEWDQQKNNVSSIIYEEIQKAQVQQEENERRLL

SEQ ID NO: 70 - protein, 34 aa, Artificial Sequence; identical to SEQ ID NO: 68
  Synthetically generated peptide
  BINDING 1: cysteic acid
  BINDING 5: organic compound

SEQ ID NO: 71 - protein, 34 aa, Artificial Sequence; identical to SEQ ID NO: 69
  Synthetically generated peptide
  BINDING 1: cysteic acid
  BINDING 8: organic compound

SEQ ID NO: 72 - protein, 34 aa, Artificial Sequenece
  Synthetically generated peptide
  BINDING 1: organic compound
WQQWERQVRFLDANITRLLEEAQIQQERNMYELQ

SEQ ID NO: 73 - protein, 34 aa, Artificial Sequence
  Synthetically generated peptide
  ACETYLATION 1
  BINDING 5: organic compound
WQQWKRQVRFLDANITRLLEEAQIQQERNMYELQ

SEQ ID NO: 74 - protein, 34 aa, Artificial Sequence
  Synthetically generated peptide
  ACETYLATION 1
  BINDING 8: organic compound
WQQWERQKRFLDANITRLLEEAQIQQERNMYELQ

SEQ ID NO: 75 - protein, 34 aa, Artificial Sequence; identical to SEQ ID NO: 73
  Synthetically generated peptide
  BINDING 1: cysteic acid
  BINDING 5: organic compound

SEQ ID NO: 76 - protein, 34 aa, Artificial Sequence; identical to SEQ ID NO: 74
  Synthetically generated peptide
  BINDING 1: cysteic acid
  BINDING 8: organic compound

SEQ ID NO: 77 - protein, 34 aa, Artificial Sequence
  Synthetically generated peptide
  BINDING 1: organic compound
WQEWEQQVRYLEANISEQLERAQIQQERNTYELQ

SEQ ID NO: 78 - protein, 34 aa, Artificial Sequence
  Synthetically generated peptide
  ACETYLATION 1
  BINDING 5: organic compound
WQEWKQQVRYLEANISEQLERAQIQQERNTYELQ

SEQ ID NO: 79 - protein, 34 aa, Artificial Sequence
  Synthetically generated peptide
  ACETYLATION 1
  BINDING 8: organic compound
WQEWEQQKRYLEANISEQLERAQIQQERNTYELQ

SEQ ID NO: 80 - protein, 34 aa, Artificial Sequence; identical to SEQ ID NO: 78
  Synthetically generated peptide
  BINDING 1: cysteic acid
  BINDING 5: organic compound

SEQ ID NO: 81 - protein, 34 aa, Artificial Sequence; identical to SEQ ID NO: 79
  Synthetically generated peptide
  BINDING 1: cysteic acid
  BINDING 8: organic compound

SEQ ID NO: 82 - protein, 34 aa, Artificial Sequence
  Synthetically generated peptide
  BINDING 1: organic compound
WQEWESQITALEGNISTTLVRAYEQEQRNMDTYQ

SEQ ID NO: 83 - protein, 34 aa, Artificial Sequence
  Synthetically generated peptide
  ACETYLATION 1
  BINDING 5: organic compound
WQEWKSQITALEGNISTTLVRAYEQEQRNMDTYQ

SEQ ID NO: 84 - protein, 34 aa, Artificial Sequence
  Synthetically generated peptide
  ACETYLATION 1
  BINDING 8: organic compound
WQEWESQKTALEGNISTTLVRAYEQEQRNMDTYQ

SEQ ID NO: 85 - protein, 34 aa, Artificial Sequence; identical to SEQ ID NO: 83
  Synthetically generated peptide
  BINDING 1: cysteic acid
  BINDING 5: organic compound

SEQ ID NO: 86 - protein, 34 aa, Artificial Sequence; identical to SEQ ID NO: 84
  Synthetically generated peptide
  BINDING 1: cysteic acid
  BINDING 8: organic compound

SEQ ID NO: 87 - protein, 36 aa, Artificial Sequence; identical to SEQ ID NO: 1
  Synthetically generated peptide
  ACETYLATION 1
  BINDING 10: organic compound

SEQ ID NO: 88 - protein, 36 aa, Artificial Sequence; identical to SEQ ID NO: 2
  Synthetically generated peptide
  ACETYLATION 1
  BINDING 3: organic compound

SEQ ID NO: 89 - protein, 36 aa, Artificial Sequence; identical to SEQ ID NO: 2
  Synthetically generated peptide
  ACETYLATION 1
  BINDING 3: organic compound

SEQ ID NO: 90 - protein, 36 aa, Artificial Sequence; identical to SEQ ID NO: 2
  Synthetically generated peptide
  BINDING 3: organic compound

SEQ ID NO: 91 - protein, 38 aa, Artificial Sequence; identical to SEQ ID NO: 3
  Synthetically generated peptide
  BINDING 38: organic compound

SEQ ID NO: 92 - protein, 34 aa, Artificial Sequence; identical to SEQ ID NO: 4
  Synthetically generated peptide
  BINDING 34: organic compound

SEQ ID NO: 93 - protein, 34 aa, Artificial Sequence; identical to SEQ ID NO: 12
  Synthetically generated peptide
  ACETYLATION 1

SEQ ID NO: 94 - protein, 36 aa, Artificial Sequence
  Synthetically generated peptide
  ACETYLATION 1
SGIVQQQNNLLRAIEAQQHLLQLTVWGIKQLQARIL

SEQ ID NO: 95 - protein, 36 aa, Artificial Sequence
  Synthetically generated peptide
  ACETYLATION 1
SGIVQQQNNLLRAIEAQQHLLQLTVWGIRQLQARIL

SEQ ID NO: 96 - protein, 36 aa, Artificial Sequence; identical to SEQ ID NO: 94
  Synthetically generated peptide